(19) United States
(12) Patent Application Publication
(10) Pub. No.: US 2024/0415750 A1
(43) Pub. Date: Dec. 19, 2024

(54) WATER REDUCED COMPACT CONDITIONER COMPOSITION

(71) Applicant: WELLA GERMANY GMBH, Darmstadt (DE)

(72) Inventors: Christian SPRINGOB, Lorsch (DE); Antje RUPPACH, Darmstadt (DE); Antonio MARTINEZ-CAMPOY, Ruesselsheim (DE)

(21) Appl. No.: 18/703,265

(22) PCT Filed: Oct. 21, 2022
(86) PCT No.: PCT/EP2022/079445

§ 371 Date: Apr. 19, 2024

(30) Foreign Application Priority Data
Oct. 22, 2021 (WO) PCT/EP2021/079369

(51) Int. Cl. A61K 8/34; A61K 8/23; A61K 8/49; A61Q 5/00; A61Q 5/12

(52) U.S. Cl. CPC A61K 8/345; A61K 8/23; A61K 8/34; A61K 8/342; A61K 8/4926; A61K 8/4933; A61K 8/4946; A61Q 5/006; A61Q 5/12

(57) ABSTRACT

A water reduced conditioner composition is disclosed. The conditioner composition comprises a polyol, a cationic surfactant, a fatty alcohol and a minimum amount of water.

### FIELD OF THE INVENTION

The present invention relates to a stable, water reduced and compact conditioner composition that comprises at least the components of one or more cationic surfactants, one or more fatty alcohols, one or more polyols and a very low concentration of water.

### BACKGROUND OF THE INVENTION

Many consumers and stylists are requesting more sustainable products that have a reductive impact on carbon dioxide emission and water footprint (concentration of water). For instance, compact/water reduced conditioner compositions can contribute to improved sustainability because conventional conditioners typically contain more than 80% of water. A conditioner with a lower water level such as a conditioner concentrate can reduce transportation weight, result in less packaging waste and deliver an overall better carbon dioxide footprint due to lower energy costs for production and transportation.

Nevertheless, today's consumers do expect high performing conditioners and are typically not willing to compromise on the product performance for improved sustainability. A typical problem with water reduced, compact conditioners is the difficulty to formulate a conditioner composition that has acceptable rinseability, conditioning performance, and stability. Especially rinseability speed and ease of dilution are often problems since water reduced, compact conditioners have a higher, pasty consistency which needs a longer, more intense and more water consuming wash-out phase.

It would therefore be of tremendous advantage to have compact water reduced conditioner compositions delivering the same or even better product performance and rinseability in comparison to conventional water-based conditioners.

### SUMMARY OF THE INVENTION

According to the present invention, it has now surprisingly been found that a conditioner composition comprising at least a cationic surfactant, a fatty alcohol, a polyol, and a very small amount of water provides a compact, significantly water reduced conditioner. This conditioner composition enables a drastically reduced concentration of water, such as but not limited to a maximum of about 10% to 20% of the usual water concentration of a conventional conditioner. The combination of the conditioning components and their weight ratio ranges delivers significant rinseability similar to that of a high water content conditioner while avoiding the disadvantages of known compact/water reduced conditioners. Additionally, the compositional embodiments of the present invention achieve the good product performance and washability/rinseablity at a lower concentration of the conditioner key ingredients such as but not limited to the cationic surfactant and the fatty alcohol compared with those of a conventional conditioner.

Components of embodiments of the conditioner composition of the present invention include at least (a) one or more polyols, (b) one or more cationic surfactants, (c) one or more fatty alcohols and (d) water. The weight percentage ranges for each of these components as well as weight percentage ranges for optional components are provided in Table 1 below.

The compositional embodiments may but not necessarily include optional components such as additional conditioning agents, antidandruff agents and direct hair dyes.

### Definitions

Unless defined otherwise, all technical and scientific terms used herein have the same meaning as commonly understood by a person of ordinary skill in the art.

As used in the specification and the appended statements, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers.

The term “about 0 wt %” is understood to mean that no substance, compound or material to which zero (0) refers is present, up to a negligible but detectable amount is present, assuming that the detectability can be determined on a parts per million basis.

Where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being ethyl and X being propyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “substantially free from” or “substantially free of” means less than 0.1%, preferably less than 0.05%, more preferably less than 0.01%, and most preferably about 0.00%, by total weight of the composition or formulation.

### BRIEF DESCRIPTION OF THE DRAWINGS

The components in the Figures are not necessarily to scale, instead emphasis being placed upon illustrating the principles of the invention. The accompanying drawings relate to embodiments of the disclosure and are described in the following:

FIG. 1 shows three images illustrating an exemplary method of conditioning hair according to embodiments described herein. FIG. 1 shows ease of rinsing off the composition according to embodiments described herein.

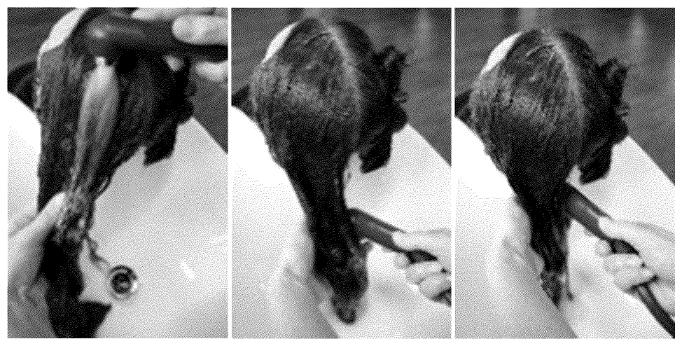

### DETAILED DESCRIPTION OF THE INVENTION

Aspects of the present invention are directed to a conditioner composition comprising a limited concentration of water and to a method for use of the conditioner composition. Embodiments of the invention deliver an easy to use, thick, flowable, dilutable, readily rinsible conditioner composition in highly concentrated form that avoids excess weight of water typical of ordinary conditioners with high water concentrations. The methods according to the invention include the ability to readily dilute the conditioner with shower water and to readily rinse off excess conditioner after its application to the hair.

Embodiments of the conditioner composition according to the invention include components of one or more polyols (a), one or more cationic surfactants (b), one or more fatty alcohols (c), water (d) and one or more optional components. The desirable conditioner composition performance properties described above may be achieved at least in part by a conditioner composition comprising balance of components b, c and d. These components are believed to provide a “creamy” formulation of a lamellar gel network. The gel network is a lipid bilayer of the mixture of fatty alcohol, cationic surfactant and water. An exemplary balance of these components suitable for development of the gel network may comprise a weight ratio of the sum of cationic surfactant (b) and fatty alcohol (c) to water (d) [b+c:d] in the range of about of about 0.1:1 to about 2:1, preferably about 0.3:1 to about 1.5:1, most preferably about 0.6:1 to about 1.3:1, especially most preferably about 0.8:1 to about 1.1:1 with a preferred exemplary ratio of about 0.87:1.

Monoalcohols (having from 1 to 7 carbon atoms) are frequently used in hair treatment compositions, and especially also in conditioner compositions. The inventors of the present disclosure have surprisingly found that the conditioning performance of water-reduced conditioner compositions is significantly improved by reducing the amount of monoalcohols as much as possible or even eliminating the monoalcohols entirely. Without being bound by theory, it is believed that the presence of monoalcohols is detrimental to the conditioning performance effect due to a negative impact of the monoalcohol (such as ethanol) on the deposition of the lubricating actives on hair (such as the cationic surfactant). It is believed that the monoalcohol (such as ethanol) enhances the wash-out of the cationic surfactant in the rinse-out step of the product application, and therefore has an obstructive effect on the performance of the cationic surfactant in conditioning the hair.

Embodiments of the conditioner composition according to the present disclosure are substantially free of monoalcohols having from 1 to 7 carbon atoms or comprise one or more monoalcohols having from 1 to 7 carbon atoms at a concentration range below 5 wt %. Preferably, the concentration of monoalcohols having from 1 to 7 carbon atoms is below 2 wt %., more preferably below 1 wt %., even more preferably below 0.5 wt %., and still more preferably below 0.4 wt %. Although omitting monoalcohols having from 1 to 7 carbon atoms entirely is most preferable, many ingredients are provided by suppliers already containing small amounts of monoalcohols. The inventors have found that the detrimental effects observed when using monoalcohols in water-reduced conditioner compositions can be largely overcome by reducing the amount monoalcohols (e.g. to 1 wt %.) without having to remove/omit the components altogether.

Embodiments of the conditioner composition according to the present disclosure preferably do not include a monoalcohol having 1 to 7 carbon atoms selected from the group consisting of methanol, ethanol, propanol, butanol, pentanol, hexanol, heptanol, iso-propanol, iso-butanol, neo-pentanol, t-butanol, 2-methyl-2-butanol, 2,3-dimethyl-2-butanol, 2-methyl-2-pentanol, 3-methyl-3-pentanol, 2,3-dimethyl-2-pentanol, 2,3-dimethyl-2-pentanol, 2,3-dimethyl-2-pentanol, 2,3-dimethyl-3-pentaol, 2-methyl-2-hexanol and 3-methyl-3-hexanol.

Embodiments of the conditioner composition according to the present disclosure are preferably substantially free of alkoxylated surfactants, such as alkoxylated fatty alcohols. Alkoxylated surfactants are considered to have an unfavourable carbon footprint and are avoided by many consumers. The present disclosure provides conditioning compositions which preferably are free of alkoxylated surfactants while providing equal or improved performance to compositions including alkoxylated surfactants. The concentration of alkoxylated surfactants may be from 0.0 wt % to 0.2 wt %, preferably from 0.0 wt % to 0.1 wt %, and more preferably from 0.0 wt % to 0.01 wt %.

The performance properties of the conditioner embodiments of the invention are also achieved by one or more polyols, preferably a polyol combination of a polyol with a higher hydroxyl count (a1) and a polyol with a lower hydroxyl count (a2). Especially preferably, this combination comprises (a1) a trihydric alcohol and (a2) a dihydric alcohol. This combination also may have a compositional weight ratio range of the trihydric alcohol component (a1) to the dihydric alcohol, component (a2).

The polyol serves as the compositional component delivering at least in part fluidity, rinseability and ease of use to the conditioner composition. The polyol may be solid or liquid under ambient conditions. When the polyol is a solid or is a liquid with a higher number of hydroxyl groups, the polyol may be combined with a liquid polyol having a lower number of hydroxyl groups and/or may be combined with a minimum amount of water to form a flowable dispersion or solution.

The polyol component of the present shampoo composition may be any one or more embodiments of a polyhydric, tetrahydric, trihydric and/or dihydric alcohol (i.e., a per-hydroxy alkane, a tetraol, a triol or a diol) or a dimer, trimer tetramer, pentamer thereof formed by condensing multiple hydroxy groups to form ether groups such as is formed by condensing two ethylene glycols to form diethylene glycol. The polyol may be liquid or solid and may be at least substantially miscible to fully miscible in water and/or in a monoalcohol such as ethanol or propanol. A polyol may alternatively be pasty at ambient conditions and may be combined with a liquid polyol to provide a flowable mixture or solution. The polyhydric alcohol may be a C4-C8 alkane with each carbon substituted by a hydroxyl group or a dimer or trimer thereof. The tetrahydric alcohol may be a C4-C8 alkane substituted by four hydroxyl groups or a dimer or trimer thereof. The trihydric alcohol may be a C3 to C8 alkane substituted by three hydroxyl groups or a dimer or trimer thereof. The dihydric alcohol may be a C2 to C8 alkane substituted by two hydroxyl groups or a dimer or trimer thereof. Preferably, the tetrahydric, trihydric and dihydric alcohols in pure form and without dilution with water display liquid and/or flowable properties at ambient conditions. Exemplary polyhydric alcohols include sugar alcohols such as but not limited to erythritol, xylitol and sorbitol. Exemplary tetraols may be selected from erythritol, tetrahydroxy pentane, tetrahydroxy hexane, tetrahydroxy heptane and/or tetrahydroxy octane of any distribution of hydroxyl groups along the alkyl chain. Exemplary triols may be selected from propylene triol (glycerin), 1,2,3-butylene triol, 1,2,5-pentylene triol and/or 1,3,6-hexylene triol and any combination thereof. Exemplary diols may be selected from ethylene glycol, 1,2-propylene diol, 1,3 propane diol, 1,2- and 1,3-butane diol, 1,2 pentane diol, diethylene glycol, dipropylene glycol and any combination thereof. Preferred polyols include sorbitol, glycerin (propyl triol), 1,2,3-butanetriol, ethylene glycol and diethylene glycol and any combination thereof. A more preferred polyol is a combination of a trihydric alcohol and a dihydric alcohol such as a combination of glycerin and ethylene glycol.

The polyol component of the present conditioner composition may have a concentration of from about 50 wt % to about 95 wt % relative to the total weight of the conditioner composition. A polyol with a higher number of hydroxyls may be combined with another polyol having a lower number of hydroxyls. Additionally, a polyol with a high carbon count may be combined with another polyol having a low carbon count. A polyol with a high carbon count usually but not necessarily may be a solid polyol while a polyol with a low carbon count usually but not necessarily may be a liquid polyol. Within these parameters involving carbon and hydroxyl counts, a solid polyol may be combined with a liquid polyol and/or a minor amount of water to provide a flowable dispersion and/or solution. It has been found that these relationships enable facile formulation of a flowable solution and/or dispersion of the conditioner composition. The higher hydroxyl polyol and lower hydroxyl polyol may be combined according to a weight ratio range of from about 0.4:1 to about 2.3:1, preferably about 0.8:1 to about 0.9:1, more preferably about 0.85:1 to about 0.87:1 wherein the ratio is higher to lower. Additionally, high and low carbon count polyols may be combined according to a weight ratio range of from about 0.1:1 to about 0.75:1 wherein the ratio is high carbon count to low carbon count. For these combinations, the higher and lower hydroxyl polyols and the high and low carbon count polyols may be individual polyols or may be mixtures of similar polyols. For example, a high carbon count polyol such as sorbitol may be combined with another high carbon count polyol such as 1,3,6-hexylene triol. A low carbon count polyol such as ethylene glycol may be combined with another low carbon count polyol such as 1,2-propylene diol. A higher hydroxyl polyol such as sorbitol may be combined with another higher hydroxyl polyol such as erythritol or glycerin. A lower hydroxyl polyol such as diethylene glycol may be combined with 1,2-propylene diol. Examples of combinations of a high carbon polyol and a low carbon polyol include sorbitol and ethylene glycol; erythritol and ethylene glycol; hexylene tetraol and ethylene glycol; glycerin and ethylene glycol.

Preferred combinations of the polyol for the present conditioner composition comprise a mixture of (a1) a higher hydroxyl count polyol such as but not limited to a trihydric alcohol and (a2) a lower hydroxyl count polyol such as but not limited to a dihydric alcohol. These combinations have a weight ratio range of component (a1), e.g., the trihydric alcohol to (a2), e.g., the dihydric alcohol (i.e., [a1:a2]) of about 0.4:1 to about 2.3:1, preferably about 0.8:1 to about 0.9:1, more preferably about 0.85:1 to about 0.87:1. Qualitatively, it has been discovered that fluid liquid to semi-fluid liquid to viscous liquid polyol mixtures of the higher hydroxyl count polyol and lower hydroxyl count polyol deliver at least in part the above described desirable conditioner composition properties. Preferably these compositional properties are achieved when the wt % concentration of the higher hydroxyl count polyol such as a trihydric alcohol is slightly to moderately less than that of the lower hydroxyl count polyol such as a dihydric alcohol in terms of weight percentage ratios.

The more preferably polyol combination for embodiments of the conditioning composition may be selected C2-C6 alkyl triols and diols that display liquid and/or flowable properties at ambient conditions. Exemplary diols may be selected from ethyl, propyl, butyl, pentyl diol of any hydroxyl group distribution along the alkyl chain, diethylene glycol, dipropylene glycol and any combination thereof. Exemplary triols may be selected from propyl, butyl and/or pentyl triol and any combination thereof. Any combination of a diol and a triol selected from this group may also fulfill the diol/triol component of embodiments of the conditioning composition. Preferred combinations include ethyl diol (ethylene glycol) and propyl triol (glycerin); 1,2- or 1,3-propyl diol and propyl or butyl triol; butyl diol and propyl triol; pentyl diol and propyl triol; pentyl diol and butyl triol having any possible hydroxyl distribution along the alkyl chain. More preferred combinations include ethyl diol (ethylene glycol) and propyl triol (glycerin); 1,2- or 1,3-propyl diol and propyl triol (glycerin); 1,2-, 1,3- and 2,3-butyl diol and propyl triol (glycerin). More preferred combinations include ethyl diol (ethylene glycol) and propyl triol (glycerin) as well as 1,2- or 1,3-propyl diol and propyl trio (glycerin)l. Most preferred combinations include ethyl diol (ethylene glycol) and propyl triol (glycerin) as well as 1,2- or 1,3-propyl diol and propyl triol (glycerin).

The cationic surfactant component of embodiments of the conditioning composition may be selected from quaternary ammonium and tertiary amine compounds, amido ammonium and tertiary amine compounds, mono, di and polysaccharides with quaternary ammonium or tertiary amine groups, or any mixture thereof. Non-limiting classes of these cationic surfactants include mono or di fatty alkyl or alkenyl quaternary ammonium and tertiary amine compounds, mono or di polyol quaternary ammonium and tertiary amine compounds, mono or di fatty alkyl or alkenyl benzyl quaternary ammonium and tertiary amine compounds, fatty alkyl or alkenyl aryl, polyol quaternary ammonium and tertiary amine compounds, polyol mono, di or tri saccharide alkyl quaternary ammonium and tertiary amine compounds, tertiary or quaternized amino alkyl fatty alkyl or alkenyl ester or amide compounds, phenoxy-alkyl fatty alkyl or alkenyl quaternary ammonium and tertiary amine compounds, hydrolyzed starch with terminally quaternized ammonium and tertiary amine compounds, multi-polyol cellulose with terminal quaternized ammonium and tertiary amine compounds, sucrose, lactose mono and disaccharides, arabic, ghatti, guaicum, guar, karaya, locust bean and xanthan gums derivatized with terminal quaternized ammonium and tertiary amine compounds, and similar hydrophobic tail with cationic head organic compounds. The classes of cationic surfactants with tertiary amine groups may be protonated by the media or by organic acid to form cationic groups.

Examples of these cationic surfactants may be selected from and/or include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamidopropyidiethylamine, arachidamidoethyidiethylamine, arachidamidoethyidimethylamine, benzalkonium chloride, benzodidecinium bromide, cetalkonium chloride, dicedyldimenthlammonium chloride dimethyldioctadecylamminum chloride, dioleoyl-3-trimethyl ammonium propane, lauryl methyl gluceth-10 hydroxylpropyl dimonium chloride N-oleyl-1,3-propane diamine, stearalkonium chloride and mixtures thereof. Additional cationic surfactants include cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride; hydrophilically substituted cationic surfactants having the following INCI designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and any combination thereof.

The fatty alcohol may be selected any C8 to C40 alkanyl mono or diol or alkenyl mono or diol. Preferably the fatty alcohol of these groups may be the mono alcohol. For the fatty alkenyl alcohols, the number of unsaturations may range from 1 to 6, preferably 1 to 3, more preferably 1 or 2, most preferably 1. Preferable fatty alcohols may be selected from C8 to C30 alkanyl or alkenyl mono alcohols. More preferably fatty alcohols may be selected from C10 to C26 alkanyl or alkenyl mono alcohols. Especially preferably fatty alcohols may be selected from C10 to C26 alkanyl mono alcohols. While it is not a requirement of the invention, the fatty alcohol may be a combination of lower and higher carbon number fatty alcohols. An exemplary combination may include a C10-C16 fatty alcohol and a C16-C22 fatty alcohol with the higher carbon number fatty alcohol constituting about a 2 to 3 fold more in wt % than the lower carbon number fatty alcohol.

According to an embodiment, the composition includes two or more fatty alcohols. Preferably the two or more fatty alcohols include at least two C16-C22 fatty alcohols, such as cetyl (palmitic) alcohol and stearyl alcohol.

Examples of these fatty alcohols may be selected from and/or include octanol, plargonic alcohol, decanol, undecyl alcohol, lauryl alcohol, tridecyl alcohol, myristyl alcohol, pentadecyl alcohol, cetyl (palmitic) alcohol, heptadecyl alcohol, stearyl alcohol, nonadecyl alcohol, arachidyl alcohol, heneicoyl alcohol behenyl alcohol, lighnocyeryl alcohol, ceryl alcohol, montanyl alcohol, nonacesanol, myricyl alcohol as well as unsaturated alcohols selected from and/or including pamitoleyl alcohol, oleyl alcohol, erucyl alcohol, arachidonic alcohol, linoleic alcohol, and farnesyl alcohol.

The water content may be maintained at a low weight percentage due to the ratio combinations and concentrations of the polyol, especially polyol combinations, cationic surfactant and fatty alcohol. This combination provides a concentrated conditioner composition that is thick, flowable, dilutable easily with water during a shampoo and conditioner treatment and rinses readily. The typical water concentration range and preferred ranges are provided in Table 1.

The compositions according to the present disclosure preferably contain a minimum amount of water, i.e. are preferably not entirely anhydrous. For example, the composition includes water at a concentration range of at least about 3 wt %, or preferably of at least about 4 wt %, and more preferably of at least about 5 wt % or even at least 6 wt % relative to the total weight of the conditioner composition. The inventors have found that anhydrous conditioning compositions have an unfavourable viscosity and are difficult to apply and distribute evenly over the hair. Application of such anhydrous compositions can take longer and can require the use of excess water to sufficiently rinse out the anhydrous conditioning composition.

The concentrations of the cationic surfactant, fatty alcohol, polyol or polyol combinations, water and optional vegetable oil, optional hydrogenated olive oil/ethyl hexyl olivate, optional direct dye, and optional anti-dandruff agent for embodiments of the conditional composition may range as given in Table 1 in weight percentages relative to the total weight of the composition.

Another aspect of the invention comprises additional optional components for inclusion in embodiments of the conditioner composition. These additional optional components may include but are not limited to smoothing, silkiness, restorative, anti-dandruff, coloration and preferably “green, environmentally beneficial” components such as vegetable oil, a hydrogenated complex of vegetable oil/ethyl hexyl fatty acid ester which preferably may be the hydrogenated complex of olive oil and ethyl hexyl olivate, anti-dandruff agent, direct dye and a solubilizing agent.

Vegetable oil provides a smoothing silky quality to hair especially hair damaged by permanent coloring and permanent curling. Vegetable oil may restore natural luster and alleviates brittleness and dryness. Hydrogenated vegetable oils are preferred due to their ability to avoid oxidation and rancidity. Any of the canola, coconut, corn, cottonseed, flaxseed, grape seed, olive, palm, peanut, safflower, sesame, soybean and sunflower oils and any mixture thereof may be included as an optional luster and anti-brittleness enhancer in the conditioner composition embodiments according to the invention. Preferred hydrogenated oils include coconut, olive, palm, safflower, sunflower, and flaxseed oils. More preferred hydrogenated oils include coconut, olive, and safflower oils. Most preferred hydrogenated oils include coconut and olive oils. The optional vegetable oil may be included in embodiments in weight percentage ranges given in Table 1.

Another optional component useful for providing a silkiness and smooth feel to hair is a complex of hydrogenated ethyl hexyl trans-esterified vegetable oil C14-C20 esters, preferably hydrogenated C16-C18 ethyl hexyl trans-esterified vegetable oil esters. An especially preferred component of this complex is hydrogenated ethyl hexyl olivate/olive oil. This complex may provide a tactile response to hair similar to that provided by silicones such as dimethicone without the need to include a difficult-to-remove silicone polymer. The complex may be formed by hydrogenating the C14-C20 vegetable oil followed by acidic transesterification with ethyl hexyl alcohol. The alcohol replaces the glycerin of the vegetable oil so as to convert the tri-ester oil into multiple mono-esters of the multiple fatty acids making up the carboxylic ester portion of the oil. The optional complex may be included in embodiments in the weight percentage ranges given in Table 1.

Additional embodiments of the conditioner composition include another optional component which is an anti-dandruff agent. Examples of the anti-dandruff agent include ketoconazole, climbazole, zinc pyrithione selenium disulfide, coal tar or sulfur. Selection of the optional components is made based on their compatibility with the cationic surfactant and the fatty alcohol. The optional anti-dandruff agent may be included in embodiments in the weight percentage ranges given in Table 1.

Another optional component for inclusion in embodiments of the conditioner composition includes direct dyes to provide coloring benefits. The technology of the present invention is suitable for nonionic direct dyes, especially for nonionic nitrobenzene derivatives and anionic direct dyes.

Examples of nitrobenzene derivatives include but are not limited to, for example, 4-hydroxypropylamino-3-nitrophenol, 1-amino-5 chloro-4-[(2,3-dihydroxypropyl)amino]-2-nitrobenzene (HC Red No. 10), and 5-chloro-1,4-[di-(2,3-dihydroxypropyl)amino]-2-nitrobenzene (HC Red No. 11) or 4-hydroxypropylamino-3-nitrophenol. Nonionic nitro dyes useful herein include, for example, 1,4-bis-[(2-hydroxyethyl)amino]-2-nitrobenzene, 1-(2-hydroxyethyl)amino-2-nitro-4-[di-(2-hydroxyethyl)amino]benzene (HC Blue No. 2), 1-amino-3-methyl-4-[(2-hydroxyethyl)amino]-6-nitrobenzene (HC Violet No. 1), 2-amino-4, 6-dinitrophenol, 1,4-diamino-2-nitrobenzene (C176070), 4-amino-2-nitrodiphenylamine (HC Red No. 1), 1-amino-4-[di-(2-hydroxyethyl)amino]-2-nitrobenzenehydrochloride (HC Red No. 13), 1-amino-5-chloro-4-[(2-hydroxyethyl)amino]-2-nitrobenzene, 4-[(2-hydroxyethyl)amino]-3-nitrophenol, 4-[(2-nitrophenyl)amino]phenol (HC Orange No. 1), 1-[(2-amino ethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-2-[(2-hydroxyethyl)amino]-5 nitrobenzene (HC Yellow No. 5), 1-(2-hydroxyethoxy)-2-[(2 hydroxyethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-methoxy-5-nitrobenzene as well as similar variations and derivatives thereof as known in the dye literature.

Other nonionic direct dyes useful herein include, for example, 1,4-di-[(2,3-dihydroxypropyl)amino]-9,10-anthraquinone, 1,4-di-[(2-hydroxyethyl)amino]-9,10-anthraquinone (C161545, Disperse Blue 23), 1-amino-4-hydroxy-9,10-anthraquinone (C160710, Disperse Red 15), 1-hydroxy-4-[(4-methyl-2-sulfophenyl)amino]-9,10-anthraquinone, 7-beta-D-glucopyranosyl-9,10-dihydro-1-methyl-9, 10-dioxo-3,5,6,8-tetrahydroxy-2-anthracenecarboxylic acid (C175470, Natural Red 4), 2-((4-(di-(2-hydroxyethyl)amino)phenyl)amino)-5-((2-hydroxyethyl)amino)-2,5-cyclohexadien-1,4-dione (HC Green No. 1), 2-hydroxy-1,4-naphthoquinone (C175480, Natural Orange No. 6), 1,2-dihydro-2-(1,3-dihydro-3-oxo-2H-indol-2-ylidene)-3H-indol-3-one (C173000), 1-[di-(2-hydroxyethyl)amino]-4-[(4-nitrophenyl)azo]benzene, (Disperse Black No. 9), 4-[(4-aminophenyl)azo]-1[di-(2-hydroxyethyl)amino]-3-methylbenzene (HC Yellow No. 7), 2), tri-(4-amino-3-methylphenyl) carbenium chloride (C142520; Basic Violet No. 2), 1-[(4-aminophenyl)azo]-7 (trimethylammonio)-2-naphthol chloride (C112250; Basic Brown No. 16), 3-[(4-amino-2,5-dimethoxyphenyl)azo]-N, N,N-trimethylbenzolaminium chloride (C11 12605, Basic Orange No. 69), 1-[(4-amino-2-nitrophenyl)azo]-7-(trimethylammonio)-2-naphthol chloride (Basic Brown No. 17), and 1-methyl-4-((methylphenyl-hydrazono)methyl)pyridinium methyl sulfate (Basic Yellow No. 87).

Other cationic direct dyes useful herein include, for example, Benzenamine, 4-[(2,6-Dichlorophenyl)(4-1mino-3,5-Dimethyl-2,5-Cyclohexadien-1-ylidene)Methyl]-2,6 Dimethyl-, Phosphate) (HC Blue No. 15), 1-(2-morpholiniumpropylamino)-4-hydroxy-9, 1 O-anthraquinone methylsulfate, and 1-[(3-(dimethylpropylaminium)propyl)amino]4-(methylamino)-9,10-anthraquinone chloride.

Anionic direct dyes useful herein include, for example, disodium bis[4-(N-ethyl-N-3-sulfonatophenylmethyl)aminophenyl]phenylmethylium (INCI name: Acid Blue 9), Benzenesulfonic acid, 2-[(9,10-Dihydro-4-hydroxy9,10-Dioxo-1-anthracenyl)amino]-5-methyl-, monosodiumsalt (INCI name: Ext. Violet 2), p-((2-Hydroxy-1-naphthyl)azo)benzenesulfonic acid sodium salt (INCI name: Orange 4), 2,2′-(1,4-Anthraquinonylenediimino)bis(5-methylbenzenesulfonic acid) disodium salt (INCI name: ACID GREEN 25), Acides 2-(2-quinoleyl) 1,3-indanedione mono, di, trisulfoniques, sodium salt (INCI name: Yellow 10), 5-amino-4-hydroxy-3-0-(phenylazo)-2,7-naphthalenesulfonic acid, disodium salt (INCI name: Acid Red 33).

The optional direct dye may be included in embodiments in the weight percentage ranges given in Table 1.

Solubilizing agents useful as optional additives to embodiments of the conditioners according to the invention provide homogeneity and avoid component partition when dissimilar components are included. The solubilizing agents may be but are not limited to polyol C8-C20 fatty acid esters from fatty acids such as caprylic, capric, lauric, myristic, palmitic, stearic, arachidic, behenic acid esters, combinations thereof and fatty acid mixtures such as a fatty acid mixture from coconut oil, palm oil and/or safflower oil as well as optional monohydric alcohols such as ethanol, isopropanol, butanol and/or isobutanol at small to very small concentrations. The alcohol part of the ester is formed from a short number of monomeric units of a polyol such as polyethylene glycol, polypropylene glycol and/or polyglycerol of 3 to 20, preferably 4 to 10 units. Preferable solubilizing agents include caprylic, capric, lauric, myristic, palmitic acid and coconut acid esters of 4-8 polyethylene glycol and 4-8 polyglycerol. More preferable agents include caprylic, capric, lauric, palmitic acid and coconut acid esters of 4 or 6 polyethylene glycol and 4-6 polyglycerol.

The optional solubilizing agent may be included in embodiments in the weight percentage ranges given in Table 1. It is to be understood that each component and each weight range given in Table 1 can be combined in any manner. For example, the water-reduced conditioner composition of the present disclosure may include one component (such as the cationic surfactant) according to the preferred range of Table 1 below, while including another component (such as the polyol) according to the more preferred range given in Table 1. Thus, the components of the water-reduced conditioner composition range may be included in any combination of the range, the preferred range, the more preferred range and the most preferred range as given in Table 1 below.

TABLE 1

| | | | | | MORE | | MOST | | | |
|---|---|---|---|---|---|---|---|---|---|---|
| | RANGE | | PREFERRED | | PREFERRED | | PREFERRED | | Preferred | |
| COMPONENT | in WT % * | | in WT % * | | in WT % * | | in WT % * | | Example | |
| Cationic | 1-3 | | 1-2 | | 1.2-1.8 | | 1.3-1.7 | | 1.4 | |
| Surfactant | | | | | | | | | | |
| Polyol^{#} | 50-95 | | 60-95 | | 65-95 | | 70-95 | | 81-83 | |
| Higher OH | 10-65 | | 10-60 | | 20-50 | | 25-50 | | 37-39 | |
| Count Polyol | | | | | | | | | | |
| and/or Combo^{#} | | | | | | | | | | |
| Lower OH | 25-75 | | 30-70 | | 30-65 | | 30-55 | | 43-45 | |
| Count Polyol | | | | | | | | | | |
| and/or Combo^{#} | | | | | | | | | | |
| Fatty Alcohol | 0.5-10 | | 1-10 | | 3-8 | | 3-5 | | 4 | |
| Water | 0.5-10 | | 1-8 | | 3-8 | | 5-8 | | 6 | |
| Optional | 0.01-0.3 | | 0.01-0.25 | | 0.01-0.2 | | 0.01-0.1 | | 0.05 | |
| Organic Acid | | | | | | | | | | |
| Optional | 0.0-2 | (0.1-2) | 0.0-1.5 | (0.1-1.5) | 0.0-1 | (0.2-1) | 0.0-0.5 | (0.2-0.5) | 0.0 | (0.3) |
| vegetable oil** | | | | | | | | | | |
| Optional | 0-8 | (2-8) | 0-7 | (3-7) | 0-6 | (3-6) | 0-5 (3-5, | | 0.0 | (3.9) |
| Hydrogenated | | | | | | | preferably | | | |
| olive oil and | | | | | | | 3.9-4.1) | | | |
| ethyl hexyl | | | | | | | | | | |
| olivate** | | | | | | | | | | |
| Optional direct | 0-5 | (0.1-5) | 0-4 | (0.1-4) | 0-3 | (0.2-3) | 0-3 | (0.5-3) | 0.0 | (0.7 |
| dye** | | | | | | | | | | |
| Optional anti- | 0-3 | (0.2-3) | 0-3 | (0.5-3) | 0-3 | (0.75-3) | 0-2.5 | (1-2.5) | 0.0 | (1.0) |
| dandruff | | | | | | | | | | |
| agent** | | | | | | | | | | |
| Optional | 0-3 | (0.1-3) | 0-2.5 | (0.2-2.5) | 0-2 | (0.2-2) | 0-2 | (0.25-2) | 0.0 | (0.3) |

Solubilization
agent**

* Each wt % is an “about” number, which is defined as ±10% of the number.
**The number range in non-parenthesis indicates a zero amount up to an added wt % amount which means inclusion is optional. If included, the wt percent range to be applied is given in parenthesis.
^{#}The Polyol may be a single polyol according to the parameters described above or a mixture of polyols according to the parameters described above. The higher and lower hydroxyl count polyols may be single polyols or a combination based on hydroxyl count as described above.

Aspects and embodiments of the invention may be defined by the following clauses.

Clause 1. A water-reduced conditioner composition comprising:
one or more polyols at a concentration range from about 50 wt % to about 95 wt %;
one or more cationic surfactants at a concentration range of from about 1 wt % to about 3 wt %;
one or more fatty alcohols at a concentration range of from about 0.5 wt % to about 10 wt %; and,
water at a concentration range of from about 0.5 wt % to about 10 wt %;
wherein the weight percentages are relative to the total weight of the conditioner composition.

Clause 2. The water-reduced conditioner composition according to clause 1 wherein the one or more polyols comprise (a1) at least one trihydric alcohol at a concentration of from about 10 wt % to about 65 wt %; and (a2) at least one dihydric alcohol at a concentration of from about 25 wt % to about 75 wt %.

Clause 3. The water-reduced conditioner composition of any of the preceding clauses wherein weight percentage ratio of the trihydric alcohol to the dihydric alcohol is in a range of from about 0.4:1 to about 2.3:1, preferably about 0.8:1 to about 0.9:1, more preferably about 0.85:1 to about 0.87:1.

Clause 4. The water-reduced conditioner composition of any of the preceding clauses wherein the sum of weight percentages of the one or more cationic surfactants and the one or more fatty alcohols to the weight percentage of the water is in a range of about of about 0.1:1 to about 2:1, preferably about 0.3:1 to about 1.5:1, most preferably about 0.6:1 to about 1.3:1, especially most preferably about 0.8:1 to about 1.1:1 with a preferred exemplary ratio of about 0.87:1.

Clause 5. The water-reduced conditioner composition of any of the preceding clauses wherein the combination of the cationic surfactant(s), fatty alcohol(s) and water form a gel network.

Clause 6. The water-reduced conditioner composition of any of the preceding clauses wherein the polyol is a trihydric alcohol comprising at least glycerin, 1,2,3-butane triol, 1,2,4-butane triol, 1,2,3-pentane triol 1,2,4-pentane triol, 1,2,5-pentane triol, 1,3,5-pentane triol, 2,3,4-pentane triol, or any combination thereof, preferably glycerin, any of the butane triols or any combination thereof; and/or a dihydric alcohol comprising at least ethylene glycol, 1,2-propylene diol, 1,3-propylene diol, 1,2-butylene diol, 1,3-butylene diol, 2,3-butylene diol, 1,4-butylene diol, 1,2-pentylene diol, 1,3-pentylene diol, 1,4-pentylene diol, 1,5-pentylene diol, 2,3-pentylene diol, 2,4-pentylene diol, 3,5-pentylene diol, diethylene glycol or dipropylene glycol or any combination thereof; preferably ethylene glycol, 1,2-propylene diol or 1,2-butylene glycol or any combination thereof; and/or preferably any combination of a trihydric alcohol and a dihydric alcohol.

Clause 7. The water-reduced conditioner composition of any of the preceding clauses wherein the cationic surfactant comprises at least a quaternary ammonium organic compound; preferably a C8-C30 mono or di fatty alkyl tri or di methyl ammonium compound, a mono or di benzyl fatty alkyl derivative thereof, a mono or di fatty alkyl polyol derivative thereof, a polyethoxylated terminal amino mono, di or poly saccharide compound or any combination thereof; more preferably a cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, benzalkonium chloride, benzodidecinium bromide, cetalkonium chloride, dicedyldimenthlammonium chloride dimethyldioctadecylamminum chloride, dioleoyl-3-trimethyl ammonium propane, lauryl methyl gluceth-10 hydroxylpropyl dimonium chloride N-oleyl-1,3-propane diamine, stearalkonium chloride, cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride; hydrophilically substituted cationic surfactants having the following INCI designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and any combination thereof.

Clause 8. The water-reduced conditioner composition of any of the preceding clauses wherein the fatty alcohol is a C8-C30 alkyl or alkenyl mono alcohol or any combination thereof; preferably a C12-C26 saturated linear alkyl mono alcohol or any combination thereof; more preferably lauryl alcohol, tridecyl alcohol, myristyl alcohol, pentadecyl alcohol, cetyl (palmitic) alcohol, heptadecyl alcohol, stearyl alcohol, nonadecyl alcohol, arachidyl alcohol, heneicoyl alcohol, behenyl alcohol or any combination thereof.

Clause 9. The water-reduced conditioner composition of any of the preceding clauses further comprising a vegetable oil, preferably a hydrogenated vegetable oil, more preferably a hydrogenated coconut, olive, safflower, sunflower, palm or flax seed oil; even more preferably a complex of a hydrogenated vegetable oil trans-esterified with ethyl hexyl alcohol, preferably a complex of hydrogenated ethyl hexyl olivate/olive oil.

Clause 10. The water-reduced conditioner composition of clause 9 comprising the vegetable oil at a concentration range of from about 0.1 wt % to about 2 wt %, preferably from about 0.1 wt % to about 1.5 wt %, more preferably from about 0.2 wt % to about 1 wt %, and most preferably from about 0.2 wt % to about 0.5 wt %.

Clause 11. The water-reduced conditioner composition of any of the preceding clauses further comprising an optional anti-dandruff agent selected from the group consisting of pirocton olamine, climbazole, ketoconazole, zinc pyrithione, selenium disulfide and mixtures thereof.

Clause 12. The water-reduced conditioner composition of clause 11 comprising the vegetable oil at a concentration range of from about 0.2 wt % to about 3 wt %, preferably from about 0.5 wt % to about 3 wt %, more preferably from about 0.75 wt % to about 3 wt %, and most preferably from about 1 wt % to about 2.5 wt %.

Clause 13. The water-reduced conditioner composition of any of the preceding clauses further comprising a direct dye selected from the group consisting of coal tar or sulfur dyes, azo dyes, anthraquinone dyes, cationic dyes, anionic dyes and mixtures thereof.

Clause 14. The water-reduced conditioner composition of clause 13 comprising the direct dye at a concentration range of from about 0.1 wt % to about 5 wt %, preferably from about 0.1 wt % to about 4 wt %, more preferably from about 0.2 wt % to about 3 wt %, and most preferably from about 0.5 wt % to about 3 wt %.

Clause 15. The water-reduced conditioner composition of any of the preceding clauses further comprising a) the one or more polyols at a concentration range from about 60 wt % to about 95 wt %, preferably from about 65 wt % to about 95 wt %, and more preferably from about 70 wt % to about 95 wt %.

Clause 16. The water-reduced conditioner composition of any of the preceding clauses further comprising b) the one or more cationic surfactants at a concentration range of from about 1 wt % to about 2 wt %, preferably from about 1.2 wt % to about 1.8 wt %, and more preferably from about 1.3 wt % to about 1.7 wt %.

Clause 17. The water-reduced conditioner composition of any of the preceding clauses further comprising c) the one or more fatty alcohols at a concentration range of from about 1 wt % to about 10 wt %; preferably from about 3 wt % to about 8 wt % and more preferably from about 3 wt % to about 5 wt %.

Clause 18. The water-reduced conditioner composition of any of the preceding clauses further comprising d) water at a concentration range of from about 1 wt % to about 8 wt %, preferably from about 3 wt % to about 8 wt % and more preferably from about 5 wt % to about 8 wt %.

Clause 19. A method of conditioning hair comprising applying either an undiluted portion or a water diluted portion of a water-reduced conditioner composition of any of the preceding clauses to the hair, massaging and/or rubbing the portion throughout the hair either as an undiluted portion or as a portion diluted with water for at least about 10 seconds, and thereafter rinsing the hair with a portion of water; and preferably optionally introducing a limited portion of water to the hair while massaging and/or rubbing the conditioner portion throughout the hair to at least in part enhance distribution of the conditioner composition throughout the hair.

Clause 20. Conditioned hair comprising hair treated according to clause 19.

Clause 21. Use of the water-reduced conditioner composition according any of the preceding clauses for reducing transportation weight and/or reducing packaging waste.

### Method for Use

Embodiments of the conditioner composition according to the invention may be applied to scalp hair through a method involving application of an aliquot of the concentrated conditioner composition directly to the hair or pre-diluting an aliquot of the concentrated composition with water followed by application to the hair. When an aliquot of the conditioner composition is applied directly, the aliquot may be distributed at least in part throughout the hair by massaging with fingers or a brush followed by dilution with water and spreading throughout the hair by rubbing and/or massaging with fingers. Working the portion of diluted composition through the hair for a period of at least 30 seconds distributes the conditioner and delivers the components as needed. The excess conditioner may be rinsed from the hair by flooding the hair with flowing warm water while rubbing the hair. The rinsed hair may be towel dried, followed by drying with a warm hair dryer while combing.

### Examples

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. The water levels can be directly added during the making process or being added as by-product in one of the raw materials, i.e. via the glycerin. Example 1.

| Raw Material | Amount | |
|---|---|---|
| Glycerin | 38.1 | g |
| Propanediol | 44.3 | g |
| Water | 6.5 | g |
| Behentrimonium Chloride | 1.4 | g |
| Cetyl Alcohol | 1.4 | g |
| Stearyl Alcohol | 3.3 | g |
| Olive Oil and Ethylhexyl Olivate, | 4 | g |
| hydrogenated | | |
| Citric Acid | 0.1 | g |
| Perfume | 0.9 | g |

| Raw Material | Amount | |
|---|---|---|
| Glycerin | 38.5 | g |
| Propanediol | 44.8 | g |
| Water | 6.6 | g |
| Behentrimonium Chloride | 1.4 | g |
| Cetyl Alcohol | 1.1 | g |
| Stearyl Alcohol | 2.6 | g |
| Olive Oil and Ethylhexyl Olivate, | 4 | g |
| hydrogenated | | |
| Citric Acid | 0.1 | g |
| Perfume | 0.9 | g |

| Raw Material | Amount | |
|---|---|---|
| Glycerin | 39.6 | g |
| Propanediol | 46.0 | g |
| Water | 6.9 | g |
| Behentrimonium Chloride | 1.8 | g |
| Cetyl Alcohol | 1.4 | g |
| Stearyl Alcohol | 3.3 | g |
| Citric Acid | 0.1 | g |
| Perfume | 0.9 | g |

| Raw Material | Amount | |
|---|---|---|
| Glycerin | 38.5 | g |
| Propane-1,3-diol | 44.76 | g |
| Water | 6.27 | g |
| Behentrimonium Chloride (in | 1.71 | g |
| Isopropyl Alcohol)* | | |
| Cetyl Alcohol | 1.1 | g |
| Stearyl Alcohol | 2.65 | g |
| Citric Acid (anhydrous) | 0.11 | g |
| Perfume | 0.9 | g |
| Olive Oil and Ethylhexyl Olivate, | 4.0 | g |

hydrogenated

*Behentrimonium Chloride (in Isopropyl Alcohol) contains 80% Behentrimonium Chloride.

### Half Head Examination of Conditioner Examples

The ability of the foregoing conditioner examples to condition the hair is examined by a procedure involving the steps of shampooing and conditioning. The procedure is conducted by shampooing the hair with shampoo that contains no conditioner and subsequently towel-drying the hair. The towel-dried hair is then parted in the middle into two sections from brow to neck. One of the sections is treated with the present invention (example 1-3), the other section is treated with the reference conditioner. This examination is termed half-head comparison because the test and reference samples are applied to one half of the head respectively, thus enabling a direct comparison under absolutely identical test conditions (identical hair structure, degree of damage, hair color etc.).

The application amount weight ratio of examples 1-3 to the reference conditioner was 0.2, the absolute application amount was 2 gram of examples 1-3 and 10 gram of the reference conditioner respectively.

The following hair care criteria were judged by experienced stylists: rinse ability, wet combability, dry combability, dry feel and anti-frizz effect.

### Evaluation Criteria for Half Head Examination:

### Rinse Ability:

Evaluation of rinse ability is determined by assessing whether a conditioner is easier or more difficult to wash out of the hair after application time. Evaluation is conducted visually by noting no visible conditioner in the hair which can be seen by lack of foam.

### Detailed Rinse Ability Measurement Protocol:

When carrying out the half-head comparison, make sure that the left and right sides are treated separately, always taking the center parting into consideration.

Ensure water force is set not too strong and water flow should be directed towards the side being rinsed.

Add a small amount of water to emulsify the product mass.

Position the shower head with flowing water at the center face line.

Use side of right hand rinse off the product on face line and the top (crown) area to the sideburn

### Rinse the Middle and Tips of Hair

If working on long hair, pile one side high at a time, so that each side can be rinsed separately.

Remove clip from section of hair to be rinsed.

After rinsing, lightly press the hair dry with a towel, taking the center parting into consideration.

Rinsing time measured is determined by the time required to rinse off the product completely on each hair section. The time required is measured with a stop watch and gives the evaluation of a longer or shorter rinsing time.

### Combability:

Combability of the hair is assessed by placing an aluminum comb parallel to the middle parting and running it through the hair to the shoulder. The comb must remain at a 900 angle throughout and remain in contact with the scalp throughout combing to avoid varying comb angles. The amount of resistance/effort needed during combing is the basis for evaluating the product as easier to comb/more difficult to comb. This test can be carried out on wet hair and on dry hair as well. The evaluation is conducted manually and is compared to the combability of the hair with reference conditioner.

### Dry Feel:

When the hair is completely dry, the evaluating stylist assesses the dry feel of the hair. This is assessed by running the hair from root to end between the thumb and middle and index fingers, while simultaneously applying light pressure; or alternatively, by running lightly outspread fingers through the hair from root to end. If the hair runs easily through the fingers, this is referred to as a smooth feel; if the hair is impeded from running easily through the fingers, this is referred to as a coarse feel.

### Frizz:

Hair Frizz is evaluated optically—on straight hair styles via assessing if short strands sticking up at the part and throughout the hair length that “project away from the main body of hair”, and on curly hair via assessing how much strands of wavy or curly hair do not align with others to form a defined wave or curl. The less hair sticking up on straight hair and the more defined/less unruly curls or waves are observed, the less frizzy the hair is.

### Examination Results

Half head examination 1 was carried out with 3 test persons, the numbers indicated how many test persons were judged for each criterion.

| | Example 1 | Example 1 | Example 1 |
|---|---|---|---|
| | better than | equal than | worse than |
| | reference | reference | reference |
| Criterion | conditioner | conditioner | conditioner |
| Rinseability | 2 | — | 1 |
| Wet Combability | 2 | — | 1 |
| Dry Feel | 1 | — | 2 |
| Dry Combability* | — | 2 | — |
| Hair Frizz | 2 | — | 1 |

*only 2 half head comparisons were carried out.

Reference conditioner composition: 82.1% water, 3.2% Behentrimonium Chloride, 3% Cetearyl Alcohol, 4% Olive Oil and Ethylhexyl Olivate hydrogenated, 2% Hydroxypropyl Starch Phosphate, 1% Phenoxyethanol and Ethylhexylglycerin, 0.8% Isopropanol, 0.5% Benzyl Alcohol, 0.5% Sodium Benzoate, 0.11% Citric Acid, 0.9% perfume, 1% Rooibos powder spray dried extract

Half head examination 2 was carried out with 3 test persons, the numbers indicated for how many test persons each criterion was judged how:

| | Example 2 | Example 2 | Example 2 |
|---|---|---|---|
| | better than | equal than | worse than |
| | reference | reference | reference |
| Criterion | conditioner | conditioner | conditioner |
| Rinseability | 2 | — | 1 |
| Wet Combability | 2 | — | 1 |
| Dry Feel | 2 | — | 1 |
| Dry Combability | 2 | 1 | — |
| Hair Frizz | 1 | 1 | 1 |

To note: the differences are statistically not significant.

Reference conditioner composition: 82.1% water, 3.2% Behentrimonium Chloride, 3% Cetearyl Alcohol, 4% Olive Oil and Ethylhexyl Olivate hydrogenated, 2% Hydroxypropyl Starch Phosphate, 1% Phenoxyethanol and Ethylhexylglycerin, 0.8% Isopropanol, 0.5% Benzyl Alcohol, 0.5% Sodium Benzoate, 0.11% Citric Acid, 0.9% perfume, 1% Rooibos powder spray dried extract

Half head examination 3 was carried out with 3 test persons, the numbers indicated for how many test persons each criterion was judged how:

| | Example 3 | Example 3 | Example 3 |
|---|---|---|---|
| | better than | equal than | worse than |
| | reference | reference | reference |
| Criterion | conditioner | conditioner | conditioner |
| Rinseability | 2 | — | 1 |
| Wet Combability | 2 | — | 1 |
| Dry Feel | 2 | — | 1 |
| Dry Combability | 2 | — | 1 |
| Hair Frizz | 1 | 2 | — |

To note: the differences are statistically not significant.

Reference conditioner composition: 82.1% water, 3.2% Behentrimonium Chloride, 3% Cetearyl Alcohol, 4% Olive Oil and Ethylhexyl Olivate hydrogenated, 2% Hydroxypropyl Starch Phosphate, 1% Phenoxyethanol and Ethylhexylglycerin, 0.8% Isopropanol, 0.5% Benzyl Alcohol, 0.5% Sodium Benzoate, 0.11% Citric Acid, 0.9% perfume, 1% Rooibos powder spray dried extract

The results of half head examination 1-3 indicates clearly that the conditioner according to the present invention leads to comparable rinseability and conditioning performance (wet and dry combability, dry hair feel and frizz reduction) despite the fact that the reference conditioner is applied with a much higher level of the key conditioning agent (cationic surfactant Behentrimonium Chloride) and with a five times higher application amount (10 g of reference conditioner vs. 2 g of the conditioner according to the present invention).

The results indicated clearly that the water reduced compact conditioner according to the present invention leads, even at a drastically reduced application amount, to a comparable conditioning performance and rinse ability in comparison to a conventional, no water reduced conditioner.

Half Head Examination of Conditioner Example 4 vs Comparative Example with Monoalcohol

The ability of the foregoing conditioner example 4 to condition the hair is examined by a half head comparison, and in comparison to a non-inventive example including monoalcohols. The comparative example is similar to example 2-H of WO/2020142514. The comparative example includes the following ingredients:

### Comparative Example

| Raw Material | Amount |
|---|---|
| Propylene Glycol | 47.44% |
| Glycerol | 0.55% |
| tricaprylate/caprate | |
| Water | 1.43% |
| Cetyl Alcohol 95% | 1.82% |
| Ethanol | 7.01% |
| Glycerin | 40.25 |
| Behentrimonium | 1.60% |
| Chloride in Isopropyl | |
| Alcohol | |

The hair of panelists is parted in the middle into two sections from brow to neck. One of the sections is treated with example 4 according to the present disclosure, the other section is treated with the comparative example.

This examination is termed half-head comparison because the test and reference samples are applied to one half of the head respectively, thus enabling a direct comparison under absolutely identical test conditions (identical hair structure, degree of damage, hair color etc.).

The following hair care criteria were judged by experienced stylists: wet and dry combability, anti-frizz and dry feel.

### Evaluation Criteria for Half Head Examination:

### Wet and Dry Combability:

Combability of the hair is assessed by placing an aluminum comb parallel to the middle parting and running it through the hair to the shoulder. The comb must remain at a 900 angle throughout and remain in contact with the scalp throughout combing to avoid varying comb angles. The amount of resistance/effort needed during combing is the basis for evaluating the product as easier to comb/more difficult to comb. This test can be carried out on wet hair and on dry hair as well. The evaluation is conducted manually and is compared to the combability of the hair with reference conditioner.

### Anti-Frizz:

Hair Frizz is evaluated optically—on straight hair styles via assessing if short strands sticking up at the part and throughout the hair length that “project away from the main body of hair”, and on curly hair via assessing how much strands of wavy or curly hair do not align with others to form a defined wave or curl. The less hair sticking up on straight hair and the more defined/less unruly curls or waves are observed, the less frizzy the hair is.

### Dry Feel:

When the hair is completely dry, the evaluating stylist assesses the dry feel of the hair. This is assessed by running the hair from root to end between the thumb and middle and index fingers, while simultaneously applying light pressure; or alternatively, by running lightly outspread fingers through the hair from root to end. If the hair runs easily through the fingers, this is referred to as a smooth feel; if the hair is impeded from running easily through the fingers, this is referred to as a coarse feel.

### Examination Results

The Half head examination was carried out with 10 test persons, the numbers indicated how many test persons were judged for each criterion.

| | Example 4 | Example 4 | Example 4 |
|---|---|---|---|
| | better than | equal to | worse than |
| | Comparative | Comparative | Comparative |
| Criterion | example | example | example |
| Wet Combability | 7 | 0 | 3 |
| Dry Combability* | 5 | 1 | 3 |
| Anti-Frizz | 8 | 0 | 2 |
| Dry Feel | 7 | 0 | 3 |

*Dry combability half head tests were carried out among nine persons.

The results of the half head examination indicate clearly that example 4 according to the present disclosure leads to a overall better conditioning performance (wet and dry combability, anti-frizz effect and dry feel) in comparison to the comparative example.

### Miscellaneous Statements

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein. The inventions, examples, results and statement of embodiments described, stated and claimed herein may have attributes and embodiments include, but not limited to, those set forth or described or referenced in this application.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed and as provided by the statements of embodiments. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims and the statements of embodiments.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims, examples and statements of embodiments. All claims and statements of embodiments including all original claims are hereby incorporated by reference in their entirety into the written description portion of the specification and the right is reserved to physically incorporated into the written description or any other portion of the application any and all such claims and statements of embodiments. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims and the statements of embodiments. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims and the statements of embodiments.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation.

The methods and processes illustratively described herein suitably may be practiced in differing orders of steps, and that they are not necessarily restricted to the orders of steps indicated herein or in the claims.

Unless defined otherwise, all technical and scientific terms used herein have the same meaning as commonly understood by a person of ordinary skill in the art.

1. A water-reduced conditioner composition comprising:
a) one or more polyols at a concentration range from about 50 wt % to about 95 wt %;
b) one or more cationic surfactants at a concentration range of from about 1 wt % to about 3 wt %;
c) one or more fatty alcohols at a concentration range of from about 0.5 wt % to about 10 wt %;
and,
d) water at a concentration range of from about 3 wt % to about 10 wt %;
e) one or more monoalcohols having from 1 to 7 carbon atoms at a concentration range of from 0 wt % to less than 5.0 wt %; and
wherein the weight percentages are relative to the total weight of the conditioner composition.

2. The water-reduced conditioner composition of claim 1 wherein the conditioner composition comprises one or more monoalcohols having from 1 to 7 carbon atoms at a concentration range of from 0 wt % to 1.0 wt %; preferably at a concentration range of from 0 wt % to 0.5 wt %.

3. The water-reduced conditioner composition according to claim 1 wherein the one or more polyols comprise (a1) at least one trihydric alcohol at a concentration of from about 10 wt % to about 65 wt %; and (a2) at least one dihydric alcohol at a concentration of from about 25 wt % to about 75 wt %.

4. The water-reduced conditioner composition of claim 3 wherein the weight percentage ratio of the trihydric alcohol to the dihydric alcohol is in a range of from about 0.4:1 to about 2.3:1, preferably about 0.8:1 to about 0.9:1, more preferably about 0.85:1 to about 0.87:1.

5. The water-reduced conditioner composition of claim 1 wherein the sum of weight percentages of the one or more cationic surfactants and the one or more fatty alcohols to the weight percentage of the water is in a range of about of about 0.1:1 to about 2:1, preferably about 0.3:1 to about 1.5:1, most preferably about 0.6:1 to about 1.3:1, especially most preferably about 0.8:1 to about 1.1:1 with a preferred exemplary ratio of about 0.87:1.

6. The water-reduced conditioner composition of claim 1 wherein the combination of the cationic surfactant(s), fatty alcohol(s) and water form a gel network.

7. The water-reduced conditioner composition of claim 1 wherein the polyol is a trihydric alcohol comprising at least glycerin, 1,2,3-butane triol, 1,2,4-butane triol, 1,2,3-pentane triol 1,2,4-pentane triol, 1,2,5-pentane triol, 1,3,5-pentane triol, 2,3,4-pentane triol, or any combination thereof, preferably glycerin, any of the butane triols or any combination thereof; and/or a dihydric alcohol comprising at least ethylene glycol, 1,2-propylene diol, 1,3-propylene diol, 1,2-butylene diol, 1,3-butylene diol, 2,3-butylene diol, 1,4-butylene diol, 1,2-pentylene diol, 1,3-pentylene diol, 1,4-pentylene diol, 1,5-pentylene diol, 2,3-pentylene diol, 2,4-pentylene diol, 3,5-pentylene diol, diethylene glycol or dipropylene glycol or any combination thereof; preferably ethylene glycol, 1,2-propylene diol or 1,2-butylene glycol or any combination thereof; and/or preferably any combination of a trihydric alcohol and a dihydric alcohol.

8. The water-reduced conditioner composition of claim 1 wherein the one or more polyols comprise glycerin at a concentration of from about 10 wt % to about 65 wt % and propanediol at a concentration of from about 25 wt % to about 75 wt %, preferably wherein the one or more polyols comprise glycerin at a concentration of from about 30 wt % to about 55 wt % and propanediol at a concentration of from about 35 wt % to about 55 wt %.

9. The water-reduced conditioner composition of claim 1 wherein the cationic surfactant comprises at least a quaternary ammonium organic compound; preferably a C8-C30 mono or di fatty alkyl tri or di methyl ammonium compound, a mono or di benzyl fatty alkyl derivative thereof, a mono or di fatty alkyl polyol derivative thereof, a polyethoxylated terminal amino mono, di or poly saccharide compound or any combination thereof; more preferably a cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamidopropyidiethylamine, arachidamidoethyidiethylamine, arachidamidoethyidimethylamine, benzalkonium chloride, benzodidecinium bromide, cetalkonium chloride, dicedyldimenthlammonium chloride dimethyldioctadecylamminum chloride, dioleoyl-3-trimethyl ammonium propane, lauryl methyl gluceth-10 hydroxylpropyl dimonium chloride N-oleyl-1,3-propane diamine, stearalkonium chloride, cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride; hydrophilically substituted cationic surfactants having the following INCI designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and any combination thereof.

10. The water-reduced conditioner composition of claim 1 wherein the fatty alcohol is a C8-C30 alkyl or alkenyl mono alcohol or any combination thereof; preferably a C12-C26 saturated linear alkyl mono alcohol or any combination thereof; more preferably lauryl alcohol, tridecyl alcohol, myristyl alcohol, pentadecyl alcohol, cetyl (palmitic) alcohol, heptadecyl alcohol, stearyl alcohol, nonadecyl alcohol, arachidyl alcohol, heneicoyl alcohol, behenyl alcohol or any combination thereof.

11. The water-reduced conditioner composition of claim 1 further comprising a vegetable oil, preferably a hydrogenated vegetable oil, more preferably a hydrogenated coconut, olive, safflower, sunflower, palm or flax seed oil; even more preferably a complex of a hydrogenated vegetable oil trans-esterified with ethyl hexyl alcohol, preferably a complex of hydrogenated ethyl hexyl olivate/olive oil.

12. The water-reduced conditioner composition of claim 11 comprising the vegetable oil at a concentration range of from about 0.1 wt % to about 2 wt %, preferably from about 0.1 wt % to about 1.5 wt %, more preferably from about 0.2 wt % to about 1 wt %, and most preferably from about 0.2 wt % to about 0.5 wt %.

13. The water-reduced conditioner composition of claim 1 further comprising an anti-dandruff agent selected from the group consisting of pirocton olamine, climbazole, ketoconazole, zinc pyrithione, selenium disulfide and mixtures thereof.

14. The water-reduced conditioner composition of claim 1 wherein the conditioner composition is substantially free of alkoxylated surfactants.

15. The water-reduced conditioner composition of claim 1 further comprising a direct dye selected from the group consisting of coal tar or sulfur dyes, azo dyes, anthraquinone dyes, cationic dyes, anionic dyes and mixtures thereof.

16. The water-reduced conditioner composition of claim 1 further comprising a) the one or more polyols at a concentration range from about 60 wt % to about 95 wt %, preferably from about 65 wt % to about 90 wt %, and more preferably from about 70 wt % to about 90 wt %.

17. The water-reduced conditioner composition of claim 1 further comprising b) the one or more cationic surfactants at a concentration range of from about 1 wt % to about 2 wt %, preferably from about 1.2 wt % to about 1.8 wt %, and more preferably from about 1.3 wt % to about 1.7 wt %.

18. The water-reduced conditioner composition of claim 1 further comprising c) the one or more fatty alcohols at a concentration range of from about 1 wt % to about 10 wt %; preferably from about 3 wt % to about 8 wt % and more preferably from about 3 wt % to about 5 wt %.

19. The water-reduced conditioner composition of claim 1 further comprising c) two or more fatty alcohols at a concentration range of from about 2 wt % to about 6 wt %; preferably from about 3 wt % to about 5 wt %.

20. The water-reduced conditioner composition of claim 1 further comprising d) water at a concentration range of from about 4 wt % to about 10 wt %, preferably from about 4 wt % to about 10 wt % and more preferably from about 5 wt % to about 8 wt %.

21. A method of conditioning hair comprising applying either an undiluted portion or a water diluted portion of a water-reduced conditioner composition of claim 1 to the hair, massaging and/or rubbing the portion throughout the hair either as an undiluted portion or as a portion diluted with water for at least about 10 seconds, and thereafter rinsing the hair with a portion of water; and preferably optionally introducing a limited portion of water to the hair while massaging and/or rubbing the conditioner portion throughout the hair to at least in part enhance distribution of the conditioner composition throughout the hair.

22. Conditioned hair comprising hair treated according to claim 21.

23. Use of the water-reduced conditioner composition of claim 1 for saving rinse water.
